(19) United States
(12) Patent Application Publication
(10) Pub. No.: US 2004/0120985 A1
(43) Pub. Date: Jun. 24, 2004

(54) Food product for increasing the cognitive functional capacity

(76) Inventor: Kurt-Reiner Geiss, Langen/Hessen (DE)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/665,394

(22) Filed: Sep. 17, 2003

Related U.S. Application Data
(63) Continuation of application No. PCT/EP02/02694, filed on Mar. 12, 2002.

(30) Foreign Application Priority Data
Mar. 26, 2001 (AT) A482/2001

(51) Int. Cl. A61K 31/70; A61K 31/685; A61K 47/00; A61K 9/68

(52) U.S. Cl. 424/439; 514/23; 514/78; 424/440

(57) ABSTRACT

A food item, preferably a bar of chocolate, that has a phosphatidyl serine content of 100 mg to 300 mg and a relatively high carbohydrate content.

### RELATED APPLICATION

This application is a continuation of International Patent Application PCT/EP02/02694 filed Mar. 12, 2002, the contents of which are here incorporated by reference in their entirety.

### BACKGROUND OF THE INVENTION

1. Field of the invention

The present invention relates to a food product, in particular, a bar, for increasing cognitive functional capacity.

2. Prior Art

It is generally known that, with increasing age, the human brain begins to lose mass, which loss can amount to as much as approximately 100 g. This loss of mass caused by, among other things, a decrease in the number of nerve cells and of the density of the synaptic connections in the neuronal network.

Scientific findings indicate that at the same time the cholesterol content in the brain increases, the phospholipids decrease. This gradual degenerative process is accompanied by a destruction of myelin and subsequently causes the phospholipid content to decrease. This, in turn, interferes with various physiological and biological cell functions.

It is generally known that these impairments have a substantial negative effect on the functional processes of the brain, in particular in the cognitive region. Thus, with increasing age, e.g., memory and the capacity to remember and to learn continuously decrease. In the terminology of the American Psychiatric Association and the American Psychological Association, this age-related decrease of the cerebral function is called or classified as Age Related Cognitive Decline (ARCD) and Age Associated Memory Impairment (AAMI), respectively.

Although the characteristics of a stable memory trace are still largely unknown, it is now assumed that the memory is stored as some type of a “biochemical change” in the system of neuronal switches. For a memory and learning process, an increased synthesis of new ribonucleic acids (RNA) is assumed. The RNA supplies the matrix for this protein synthesis.

When the phospholipid content decreases in the course of the age-related degeneration process, the membrane structure of the cellular walls which constitute the solvent medium for these proteins is gradually being destroyed. As a result, the construction of a new memory trace (short-term memory) is made more and more difficult.

Thus, the problem to be solved by the present invention is to make available a food product, preferably a bar, which increases the cognitive functional capacity, in particular in individuals who are more than 40 years old.

### SUMMARY OF THE INVENTION

The problem underlying this invention is solved by the characteristics of the food or bar described herein. Preferred embodiments are presented to illustrate the inventive concept.

The starting point of the present invention is the discovery that, in older individuals, the intake of 100 mg to 300 mg of phosphatidyl serine per day can lead to an improvement of the cognitive functional capacity, in particular the memory and learning capacity, and to an increase in the powers of concentration and attentiveness.

Phosphatidyl serine is a phospholipid and, as a nutrient, is a member of the group of lecithins. As a number of scientific studies have shown, phosphatidyl serine has specific effects on the nerve tissue, in particular in the brain. The most important function of phosphatidyl serine in the nerve tissue relates to the development of proteins in the cell membrane matrix.

These protein structures in the cell membrane are responsible for all of the important switching functions on the surface of the cell.

Thus, the transmission of signals or the communication between the cells of the brain is ensured, and thereby the prerequisite for an optimum cognitive functional capacity is created.

The phospholipid deficiency in advanced age is largely attributable to two different causes. As it is known from the hypothesis proposed by Crook T H, Adderly B (1998), “The memory cure,” New York: Pocket Books, the human body, from the standpoint of evolution, is not programmed for life at an old age. Although the quantity of phosphatidyl serine ingested with normal food is adequate up to middle age (approximately up to 45 years of age which, in prehistoric time, corresponds to one entire generation), it is no longer sufficient in advanced age.

Another reason, however, is the fact that eating habits have changed: Since the focus is now on a fat- and cholesterol-conscious diet, the consumption of food products containing phosphatidyl serine (food products of animal origin) has been considerably restricted. This means that the diet consumed now provides approximately 200 to 400 mg less phosphatidyl serine per day.

Because of this, the present invention proposes to prevent a phospholipid deficiency in older individuals by substituting or supplementing the intake of phosphatidyl serine in food. Scientific studies have demonstrated that the cognitive functional capacity of individuals older than 40 years is increased when they consume 100 mg to 300 mg of phosphatidyl serine per day.

Phosphatidyl serine has a specific nutrient-physiological effect. At these doses, a nutrition- and/or situation-induced phosphatidyl serine deficiency can be compensated for and can be made to return to the normal range by means of a targeted administration [of the nutrient]. By adding phosphatidyl serine to a food product, the present invention is creating a so-called “functional food” which, beyond the purely nutritional purpose, also has positive physiological effects with respect to the cognitive functional capacity.

Preferably, the food product according to the present invention has a relatively high carbohydrate content, such as fructose syrup, sugar and/or glucose syrup. By specifically combining the intake of carbohydrates and phosphatidyl serine, the glucose intake, and thus the glucose content in the brain cells, is markedly increased. In the short term, this makes possible an especially marked increase in the cognitive functional capacity. The minimum quantity of carbohydrates is preferably 15 g combined with preferably 100 to 300 mg of phosphatidyl serine.

Preferably, the new food product according to the present invention has the form of a bar, preferably a chocolate bar. The nutrient-physiological active ingredient in the bar is phosphatidyl serine, preferably made from a lecithin extract containing phosphatidyl serine.

In addition, the bar preferably has a relatively high carbohydrate content so as to ensure the desired combination effect of a short-term improvement of the cognitive functional capacity after consumption of phosphatidyl serine. The carbohydrate content should be higher than 40 wt %, preferably higher than 57 wt %. This corresponds to a content of more than 1 wt %, preferably 1.4 wt %, of lecithin extract containing phosphatidyl serine.

In addition, the bar preferably has a protein content of at least 10 wt %, preferably 16 wt %, and a fat content of a minimum of 15 wt %, preferably 27 wt %. Furthermore, the bar can be enriched with vitamins.

The coating of chocolate, preferably milk chocolate, increases the enjoyment function.

The product size of the bar is preferably at least 20 g, in particular 35 g. The bar can be marketed wrapped in individual packages, which simplifies handling. The intake of one bar per day suffices to ensure the lasting improvement of the cognitive functional capacity; however, it is also possible to eat several bars per day. When consumed regularly, three to four bars per week suffice to sustain a long-term increase of the cognitive functional capacity.

The bar preferably has a water content of less than 3%, which increases the stability of phosphatidyl serine.

Thus, the shelf life can be more than one year during which the nutrient-physiological effect of phosphatidyl serine is completely maintained.

### BRIEF DESCRIPTION OF THE DRAWINGS

A preferred embodiment of the invention will be explained below with reference to the drawings.

FIG. 1 shows the molecular structure of phosphatidyl serine, and

FIGS. 2A and 2B shows the composition of a preferred embodiment of the bar according to the present invention.

### DETAILED DESCRIPTION OF PREFERRED EMBODIMENTS OF THE INVENTION

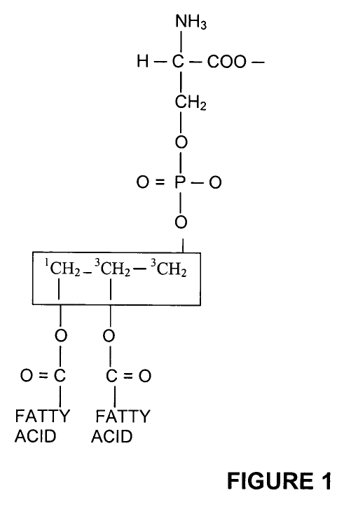

FIG. 1 shows the molecular structure of phosphatidyl serine.

Phosphatidyl serine is a member of the group of phospholipids. The phospholipids are divided into four groups, the lecithins, the kephalins, the phosphatidyl inosites, and the sphingomyelins. The glycerol phospholipids of the lecithin and kephalin group have the same structure: The first primary and the secondary hydroxyl group of the glycerol is esterified with a fatty acid, the second primary hydroxyl group is esterified with phosphate.

The phosphate in turn is esterified with a hydroxyamino compound; here, the nitrogen bases choline, ethanolamine, and serine are found. Phosphatidyl serine is a member of the group of kephalins and is therefore also known by the name of “serine kephalin.”

Chemically speaking, phosphatidyl serine thus consists of fatty acids, glycerol, phosphate, and serine.

Apart from the production of phosphatidyl serine from bovine brain, the following two preferred manufacturing methods are used: 1. Concentration of the small quantity of phosphatidyl serine of 0.2 to 0.3% that is naturally contained in soy lecithin by means of extraction and subsequent chromatographic purification. This method is, however, extremely time-consuming and expensive and requires a large number of organic solvents. 2. Enzymatic conversion of the phospholipids, phosphatidyl choline and phosphatidyl ethanolamine, contained in commercially available lecithin into phosphatidyl serine without the use of organic solvents. This method is based on the principle of enzymatic transesterification, similar to the method that is already commercially carried out for transesterifying triglycerides (e.g., cocoa butter substitutes or medium-chain triglycerides). The transesterification of fats is a method of changing the physical properties of a fat which is common practice in food technology and is widely used, e.g., for the following purposes: production of fat components with specific melting properties (consistency) for margarine compositions, baking fats and baking fat components, and for confectionery fats.

As a matter of priority, the enzymatic conversion method is preferably used.

From DE 199 172 49, a method of producing phosphatidyl serine is known in which lecithin is dispersed in water. Phospholipase D and calcium chloride, dissolved in water, are introduced into the dispersion. After stirring for 10 to 20 hours at room temperature, the calcium salt of the phosphatidyl serine is separated from the aqueous phase, and the free L-serine and choline in the phosphatidyl serine are washed out. By means of a final ethanol extraction, phosphatidyl serine and products enriched with phosphatidyl serine without residual enzyme activity are obtained.

In addition, a method for the production of phosphatidyl serine with an unsaturated fatty acid as a side chain is known from U.S. Pat. No. 5,965,413. The possibility of using phosphatidyl serine in principle to increase the cognitive functional capacity is also known from U.S. Pat. No. 5,900,409 and U.S. Pat. No. 6,117,853 as such.

Based on animal experimental studies, the following statements can be considered confirmed facts. The administration of phosphatidyl serine protects against neuron atrophy, partially prevents the age-related breakdown of the receptors for the nerve growth factor, promotes the formation of nerve growth factors (an effect specific to phosphatidyl serine which was not identified for other phospholipids), normalizes the cholesterol/phospholipid ratio in the aging brain, improves the ATPase-dependent ion transport via the cell membrane, and normalizes the protein kinase C balance.

In addition, the bioavailability of orally administered phosphatidyl serine is good (after oral administration, radioactively labeled phosphatidyl serine is detectable in the blood after 30 minutes and, after passing through the liver, subsequently crosses the blood-brain barrier).

Furthermore, neurophysiological test methods have been able to demonstrate the increase in cognitive functional capacity in subjects between 40 and 80 years old who are suffering from so-called Age Related Cognitive Decline (ARCD) and so-called Age Associated Memory Impairment (AAMI).

The tests listed below constitute a selection of the testing procedures used to demonstrate the increase in the cognitive functional capacity:

a) Demonstration of the increase in attentiveness and concentration: Diller L et al. (1974): Studies in Cognition and Rehabilitation in Hemiplegia (Letter Cancellation Test). Rehabilitation Monograph No. 50, Institute of Rehabilitation Medicine, New York, University Medical Center. Smith, A (1973): Symbol Digit Modalities Test, Los Angeles: Western Phosphatidylserinychological [sic; Psychological] Services

Wechsler D et al. (1955): Adult Intelligence Scale Manual (Digit Symbol and Digit Span (Forward/Backward)), New York: Phosphatidylserinychological Corporation

Wechsler D (1970): Echelle d'intelligence des [sic; de] Wechsler pour adultes [Wechsler Adult Intelligence Scale], WAIS, 2nd edition, Paris, Center of Applied Phosphatidylserinychology.

b) Demonstration of the increase in the memory and learning capacity:

Rey 15-Word Test for short and long-term verbal memory, Rey A (1964): L'examen Clinique en Phosphatidylserinychologie [Clinical Examination in Psychology] (Rey 15-Word Test for short and long-term verbal memory), Paris: Presses Universitaires de France; Block Tapping Test (BTT); Milner B (1971): Interhemispheric differences in the localization of Phosphatidylserinychological processes in men (Block Tapping Test (BTT)). British Medical Bulletin 27: 272; SET Test, Isaacs B et al. (1972): The Set Test, a Rapid Test of Mental Function in Old People. Age and Agening [sic; Aging] 1:222

The five words from the Randt Memory Test, Randt C T et al. (1980): A memory test for longitudinal measurement of mild to moderate deficits (The five words from Randt Memory Test). Clinical NeuroPhosphatidylserinychology [sic; Neuropsychology] 2:184.

In addition, in most of the studies available, the behavior test using the Plutchik Geriatric Rating Scale was carried out (Plutchik R et al. (1970): Reliability and validity of a scale for assessing the functioning of geriatric patients (Plutchik Geriatric Rating Scale). Journal of the American Geriatric Society 18(6):491-500.

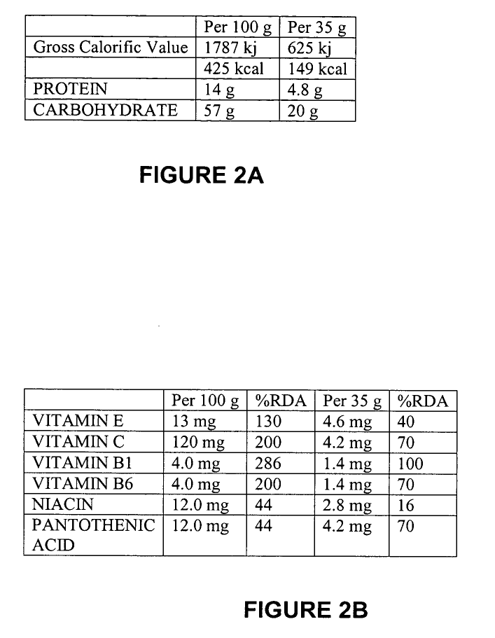

FIGS. 2A and 2B show, respectively, Tables 20 and 21 that relate to an embodiment of the chocolate bar according to the present invention. Table 20 lists the gross caloric value per 100 g and per 35 g of the chocolate bar as well as the amount of protein, carbohydrate, and fat.

Table 21 lists the composition of the chocolate bar with respect to vitamins E, C, B1, B6 and with respect to niacin and pantothenic acid.

The ingredients per 100 g of the chocolate bar are: fructose syrup, sugar, powdered skim milk, cocoa butter, powdered milk, milk protein, sweet whey powder, dextrose, hydrogenated vegetable oil, cocoa mass, maltodextrin, modified starch, rice extrudate, 1.4 g of lecithin extract, coffee extract, flavor compound, emulsifier lecithins, 120 mg of vitamin C, dried egg albumin, 13.2 [mg] of pantothenate, 13 mg of vitamin E, 8 mg of niacin, 4 mg of vitamin B1, 4 mg of vitamin B6, and 200 mg of phosphatidyl serine from lecithin extract. The product size of the bar is preferably 35 g.

Eating one or more of the chocolate bars every day in the short term leads to an increase in the cognitive functional capacity after consumption of the bar, on the one hand, and in the long term to a lasting improvement of the cognitive functions which begins to be noticeable, for example, after a period of one to three weeks. Thus, improvements both with respect to ARCD and with respect to AAMI can be reached.

The invention is, however, not restricted to bars; instead, the intake of phosphatidyl serine in a quantity of approximately 100 to 300 mg per day can take place by means of other food, in particular by means of so-called “functional food” products, for example, beverages, bread spreads, chocolate and candy products, milk, dairy products, dietetic foods, cereals, etc. Such foods should preferably have a relatively high carbohydrate content so as to obtain the desired combination effect with phosphatidyl serine with respect to the short-term increase in the cognitive functional capacity by increasing the glucose level in the brain.

What is claimed is:

1. A food for increasing the cognitive functional capacity containing a minimum of 100 mg of phosphatidyl serine, and with a minimum of 15 g of carbohydrates.

2. A food according to claim 1 wherein the phosphatidyl serine is present in an amount of from 200 to 300 mg.

3. A method comprising consuming the food of claim 1 to prevent and protect against attention deficit disorders and lack of concentration, to prevent and protect against impaired memory and learning disorders, to improve attentiveness and concentration, to improve memory and learning ability and/or to use in learning and training processes.

4. The food of claim 1 which is a “functional food” product, for example, beverages, bread spreads, chocolate products, candy products, milk, dairy products, diet foods, and cereals.

5. A bar for increasing the cognitive functional capacity with a minimum of 100 mg of phosphatidyl serine, preferably 200 mg to 300 mg of phosphatidyl serine, and with a minimum of 15 g of carbohydrates.

6. The bar of claim 5 with a weight of at least 20 g, preferably 35 g.

7. A bar for increasing the cognitive functional capacity with a minimum of 40 wt % carbohydrates, preferably 57 wt % carbohydrates, and with a minimum of 1 wt %, preferably 1.4 wt % lecithin extract containing phosphatidyl serine.

8. The bar of claim 5 with a minimum of 10 wt % protein, preferably 16 wt % protein.

9. The bar of claim 5 which is enriched with vitamins and which has a chocolate coating.

10. The bar of claim 5 with a minimum of 15 wt % fat, preferably 27 wt % fat.

11. The bar of claim 5 with a water content of less than 3%.

12. The bar of claim 5 to prevent and protect against attention deficit disorders and lack of concentration, to prevent and protect against impaired memory and learning disorders, to improve attentiveness and concentration, to improve memory and learning ability and/or to use in learning and training processes.

13. The bar of claim 5 which is a “functional food” product.
